(19) United States
(12) Patent Application Publication
(10) Pub. No.: US 2024/0175860 A1
(43) Pub. Date: May 30, 2024

(54) GLYCOMETABOLOMICS-RELATED BIOMARKERS FOR EARLY DIAGNOSIS AND PREDICTION OF DIABETIC RETINOPATHY PROGRESS AND APPLICATION THEREOF

(71) Applicant: JOINT SHANTOU INTERNATIONAL EYE CENTER OF SHANTOU UNIVERSITY AND THE CHINESE UNIVERSITY OF HONG KONG, Shantou (CN)

(72) Inventors: Mingzhi ZHANG, Shantou (CN); Xin WEN, Shantou (CN); Qingping LIU, Shantou (CN)

(21) Appl. No.: 18/520,282

(22) Filed: Nov. 27, 2023

(30) Foreign Application Priority Data
Nov. 29, 2022 (CN) 202211507791.8

(51) Int. Cl. G01N 33/487; G01N 30/72

(52) U.S. Cl. CPC G01N 33/487; G01N 30/72; G01N 2030/027

(57) ABSTRACT

Glycometabolomics-related biomarkers for early diagnosis and prediction of diabetic retinopathy and the application thereof are provided in the present disclosure, belonging to the field of biomarkers. In the present disclosure, tear fluid is used as a biological sample, which is analysed through non-invasive sampling to identify glycometabolomics-related biomarkers that are capable of being used to diagnose and predict the progression of diabetic retinopathy.

### CROSS-REFERENCE TO RELATED APPLICATIONS

This application claims priority to Chinese Patent Application No. 202211507791.8, filed on Nov. 29, 2022, the contents of which are hereby incorporated by reference.

### TECHNICAL FIELD

The present disclosure relates to the field of biomarkers, and in particular to glycometabolomics-related biomarkers for early diagnosis and prediction of diabetic retinopathy progress and an application thereof.

### BACKGROUND

Diabetic retinopathy (DR) is a microvascular ocular complication of diabetes mellitus (DM) and is the leading cause of visual impairment and irreversible blindness in the working-age population (20-65 years). The global prevalence of diabetic retinopathy in the diabetic population is 34.6%. The end-stage of diabetic retinopathy is proliferative diabetic retinopathy (PDR), with features of neovascularization, vitreous haemorrhage and preretinal haemorrhage, which seriously threaten and impair the patients' visual function.

Currently, blood glucose and glycated haemoglobin levels are still important factors affecting the progression of diabetic retinopathy; these two factors are commonly used to clinically diagnose diabetic retinopathy and predict the progression of diabetic retinopathy in combination with imaging indicators; however, such an approach has the following drawbacks: firstly, lack of convenient measures for early screening of diabetic retinopathy: studies have shown that ¾ of the diabetic population will develop diabetic retinopathy within 10 years, while early diagnosis of diabetic retinopathy mainly relies on expensive imaging examinations, such as fundus photography, fundus fluorescence angiography, and optical coherence tomography (OCT), which require patients to travel to a healthcare facility for screening, and fail to facilitate real-time monitoring of the progression of DR; secondly, lack of specific metabolic markers reflecting the progression of diabetic retinopathy: though the blood glucose and glycated haemoglobin levels affect the development of diabetic retinopathy; some DM patients would still develop DR even under tight glycemic and blood pressure control. It is found that the occurrence and development of diabetic retinopathy is not only related to glucose level but also to other glucose-related metabolites, such as lactate and fructose, according to the in-depth study of glycometabolomics; therefore, the monitoring of glycometabolomics-related markers need to be strengthened so as to facilitate the early diagnosis and delay the progression of diabetic retinopathy; thirdly, both of these biochemical indicators are currently monitored in an invasive manner, causing certain pain to the patient, making it necessary to explore the biomarkers capable of non-invasive monitoring and early diagnosis and prediction of diabetic retinopathy.

Tear fluid, as a novel biological fluid, has become a hot topic of researches lately, and is similar to but simpler than blood components in terms of composition; judging from the association of metabolites, there is a definite linear positive correlation between tear glucose and blood glucose concentration in patients with diabetic retinopaty, despite the low degree of correlation. Although there is a correlation between metabolites in blood and tear fluids, other metabolites are also involved in the occurrence and development of diabetic retinopathy. Therefore, the present disclosure investigates one or several non-invasive biomarkers against tear fluid that are capable of better diagnosing and predicting the progression of diabetic retinopathy.

### SUMMARY

It is an objective of the present disclosure to provide glycometabolomics-related biomarkers for early diagnosis and prediction of diabetic retinopathy progress and an application thereof, so as to solve the problems existing in the prior art. It is possible to differentiate the proliferative diabetic retinopathy from the non-proliferative diabetic retinopathy with high sensitivity and specificity when using lactate or a combination of lactate and glucose, fructose, galactose and mannose.

In order to achieve the above objectives, the present disclosure provides the following technical scheme.

The present disclosure provides glycometabolomics-related biomarkers for early diagnosis and prediction of diabetic retinopathy progress, and the glycometabolomics-related biomarkers include lactate, or a combination of lactate with at least one monosaccharide.

Optionally, monosaccharides include glucose, fructose, galactose and mannose.

The present disclosure also provides an application of the glycometabolomics-related biomarkers in preparing reagents or kits for early diagnosis and prediction of diabetic retinopathy, and the glycometabolomics-related biomarkers include any one of followings:
(1) lactate; and
(2) a combination of lactate with at least one monosaccharide.

The present disclosure also provides an application of reagents for detecting the glycometabolomics-related biomarkers in preparing the reagents or kits for early diagnosis and prediction of diabetic retinopathy progress, where the glycometabolomics-related biomarkers include any one of followings:
(1) lactate; and
(2) a combination of lactate with at least one monosaccharide.

Optionally, the reagents or kits for early diagnosis and prediction of diabetic retinopathy progress include reagents or kits for detecting the glycometabolomics-related biomarkers in biological samples based on Ultra-performance liquid chromatography-tandem mass spectrometry (UPLC-MS/MS).

Optionally, the biological samples include tear fluid.

Optionally, a content of at least one of the glycometabolomics-related biomarkers in the biological samples is linearly and positively correlated with a content of glucose in blood.

Optionally, the monosaccharides include glucose, fructose, galactose and mannose.

Optionally, the diabetic retinopathy includes proliferative diabetic retinopathy and non-proliferative diabetic retinopathy.

The present disclosure achieves the following technical effects.

The present disclosure uses tear fluids as biological samples to screen for glycometabolomics-related biomarkers for the early diagnosis and prediction of diabetic retinopathy, and experimentally validates that the sensitivity and specificity of the biomarker lactate or lactate combined with monosaccharides (glucose, fructose, galactose, and mannose) in tear fluid for diagnosing and predicting the progression of diabetic retinopathy are higher than those of blood glucose or a particular monosaccharide in tear fluid; thus, the present disclosure provides biomarkers for early monitoring of the progression of diabetic retinopathy in a non-invasive manner, which is important for reducing or minimizing the damage caused by diabetic retinopathy.

### BRIEF DESCRIPTION OF THE DRAWINGS

For a clearer description of the technical schemes in the embodiments or prior art of the present disclosure, the accompanying drawings to be used in the embodiments are briefly described hereinafter, and it is obvious that the accompanying drawings in the description hereinafter are only some of the embodiments of the present disclosure, and that for the person of ordinary skill in the field, other accompanying drawings are available on the basis of the accompanying drawings without any creative efforts.

FIG. 1A is a standard curve for glucose detection.

FIG. 1B is a standard curve for fructose detection.

FIG. 1C is a standard curve for galactose detection.

FIG. 1D is a standard curve for mannose detection.

FIG. 1E is a standard curve for lactate detection.

FIG. 2A shows levels of glucose in tear fluids of diabetic retinopathy patients, and non-diabetic control subjects.

FIG. 2B shows levels of fructose in tear fluids of diabetic retinopathy patients and non-diabetic control subjects.

FIG. 2C shows levels of galactose in tear fluids of diabetic retinopathy patients and non-diabetic control subjects.

FIG. 2D shows levels of mannose in tear fluids of diabetic retinopathy patients and non-diabetic control subjects.

FIG. 2E shows levels of lactate in tear fluids of diabetic retinopathy patients and non-diabetic control subjects.

FIG. 3A shows a linear relationship between fasting blood glucose levels and glucose levels in fasting tear fluid.

FIG. 3B shows a linear relationship between fasting blood glucose levels and fructose levels in fasting tear fluid.

FIG. 3C shows a linear relationship between fasting blood glucose levels and galactose levels in fasting tear fluid.

FIG. 3D shows a linear relationship between fasting blood glucose levels and mannose levels in fasting tear fluid.

FIG. 3E shows a linear relationship between fasting blood glucose levels and lactate levels in fasting tear fluid.

FIG. 4A shows a comparison of the receiver operator characteristic (ROC) curves of fasting blood glucose levels in the diabetic retinopathy population with non-diabetic control groups.

FIG. 4B shows a comparison of ROC curves for single metabolite concentration of glucose in the fasting tear fluids of the diabetic retinopathy population versus non-diabetic controls.

FIG. 4C shows a comparison of ROC curves for single metabolite concentration of fructose in the fasting tear fluids of the diabetic retinopathy population versus non-diabetic controls.

FIG. 4D shows a comparison of ROC curves for single metabolite concentrations of galactose in the fasting tear fluids of the diabetic retinopathy population versus non-diabetic controls.

FIG. 4E shows a comparison of ROC curves for single metabolite concentrations of mannose in the fasting tear fluids of the diabetic retinopathy population versus non-diabetic controls.

FIG. 4F shows a comparison of ROC curves for single metabolite concentrations of lactate in the fasting tear fluids of the diabetic retinopathy population versus non-diabetic controls.

FIG. 4G shows a comparison of ROC curves of a combination levels of glucose, fructose, galactose, mannose, and lactate in fasting tear fluids of the diabetic retinopathy population with that of non-diabetic control groups.

FIG. 4H shows a comparison of the receiver operator characteristic (ROC) curves of fasting blood glucose levels in the non-proliferative diabetic retinopathy population with proliferative diabetic retinopathy group.

FIG. 4I shows a comparison of ROC curves for single metabolite concentrations of glucose in the fasting tear fluids of the non-proliferative diabetic retinopathy population versus proliferative diabetic retinopathy group.

FIG. 4J shows a comparison of ROC curves for single metabolite concentrations of fructose in the fasting tear fluids of the non-proliferative diabetic retinopathy population versus proliferative diabetic retinopathy group.

FIG. 4K shows a comparison of ROC curves for single metabolite concentrations of galactose in the fasting tear fluids of the non-proliferative diabetic retinopathy population versus proliferative diabetic retinopathy group.

FIG. 4L shows a comparison of ROC curves for single metabolite concentrations of mannose in the fasting tear fluids of the non-proliferative diabetic retinopathy population versus proliferative diabetic retinopathy group.

FIG. 4M shows a comparison of ROC curves for single metabolite concentrations of lactate in the fasting tear fluids of the non-proliferative diabetic retinopathy population versus proliferative diabetic retinopathy group.

FIG. 4N shows a comparison of ROC curves of a combination levels of glucose, fructose, galactose, mannose, and lactate in fasting tear fluids of the non-proliferative diabetic retinopathy population with that of proliferative diabetic retinopathy group.

### DETAILED DESCRIPTION OF THE EMBODIMENTS

Various exemplary embodiments of the present disclosure are now described in detail, which detailed description should not be considered as a limitation of the present disclosure, but should be understood as a rather detailed description of certain aspects, features and embodiments of the present disclosure.

It is to be understood that the terms described in the present disclosure are only intended to describe particular embodiments and are not intended to limit the disclosure. Further, for the range of values in the present disclosure, it is to be understood that each intermediate value between the upper and lower limits of the range is also specifically disclosed. Each smaller range between any stated value or intermediate value within a stated range and any other stated value or intermediate value within said range is also included within the present disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes can be made to the specific embodiments of the present disclosure without departing from the scope or spirit of the present disclosure. Other embodiments will be apparent to the skilled person from the description of the disclosure. The specification and embodiments of this application are only exemplary.

The terms “including”, “comprising”, “having” and “containing” used in this specification are all open terms, which means including but not limited to.

Embodiment 1 Screening and identification of glycometabolomics-related biomarkers for early diagnosis and prediction of diabetic retinopathy (DR)

### 1. Determination of Research Objects

In this study, 21 non-diabetic control subjects, 20 non-proliferative diabetic retinopathy (NPDR) subjects and 19 proliferative diabetic retinopathy (PDR) subjects are enrolled from Joint Shantou International Eye Center of Shantou University and the Chinese University of Hong Kong. The diagnosis of diabetic retinopathy patients is based on the guidelines issued by American Academy of Ophthalmology (AAO) in 2019, and the eye manifestations of each patient are jointly diagnosed by two retinal specialists, and only those with consistent diagnosis are to be included. This study is approved by the committee on ethics in human medical research of the Joint Shantou International Eye Center of Shantou University and the Chinese University of Hong Kong, which is in line with the principles of Declaration of Helsinki. After explaining the nature and possible consequences of the study, all the subjects are informed and agreed.

### 2. Tear Fluids Collection

All tear samples are collected using Schirmer strips. The strips were placed in the conjunctival sac of the study subject for 10 minutes (min) and then gently removed with sterile forceps. To avoid tear evaporation, the strips were placed into 2-mL tubes and immediately stored at −80 degrees Celsius (° C.) before further processing. One strip was applied for each of the two eyes for each study subject, and the two strips from the two eyes for each subject were pooled together as one sample for liquid chromatography (LC)-mass spectrometry (MS)/MS analysis.

### 3. Sample Processing

(1) 800 microliters (μL) of 80% methanol water (methanol:water=80:100, volume to volume (v:v)) is added into the sample, followed by vortex mixing for 5 min, grinding for 180 seconds (s) at 65 hertz (Hz), standing at 4° C. for 1 hour (h), and centrifuging at 4° C. for 15 min;
(2) 200 μL of the supernatant is added into a 1.5 mL centrifuge tube, and 100 μL of 200 millimolars (mM) 3-Nitrophenylhydrazine (3-NPH) solution and 120 mM 1-ethyl-(3-dimethylaminopropyl)-carbodiimide (EDC) containing 6% pyridine are added respectively, followed by vortexing for 1 min (total volume of 400 μL) to mix evenly, and reacting at 40° C. for 1 h while shaking once every 5 min during this period;
(3) after the reaction is completed, centrifuge at 12000 revolutions per minute (rpm) at 4° C. is carried out for 15 min, and the supernatant is taken for liquid chromatograph mass spectrometer/mass spectrometry/(LC-MS/MS) analysis.

### 4. Instrument Parameters

### 4.1 Instruments: Liquid Chromatography Waters Acquity Ultra-High Performance Liquid Chromatography (UPLC), MS AB SCIEX 5500 QQQ-MS.

Chromatographic column: Acquity UPLC HSS T3 (1.8 μm, 2.1 mm*100 mm).

4.2 Ultra-High Performance Liquid Chromatography Coupled with Triple Quadrupole Mass Spectrometry (UPLC-QQQ-MS) Method:
chromatographic separation conditions: column temperature: 40° C., flow rate: 0.30 mL/min;
composition of mobile phase: A-water (0.01% formic acid) and B-acetonitrile (0.01% formic acid); and
running time: 5 min, sample volume: 6 L, and the sample gradient elution procedure is shown in the table below.

TABLE 1
Sample gradient elution procedure

| Time | Flow Rate | % B |
|---|---|---|
| Initial | 0.300 | 40.0 |
| 5.00 | 0.300 | 40.0 |

### 4.3 Mass Spectrometry Conditions:

ion source: electrospray ionization (ESI) ion source;
curtain gas: 20 arbitrary unit (arb);
collision gas: 9 arb;
ionspray voltage: −4200 volts (V);
ion source temperature: 450° ° C.;
ion source gas 1: 35 arb;
ion source gas 2: 35 arb.

### 4.4 Multiple Reaction Monitoring (MRM) Acquisition Parameters

According to the chromatographic and mass spectrometry conditions illustrated above and in Table 1, the prepared standard solution is added into the sample bottle for injection; see Table 2 for the retention time of substance peak.

TABLE 2
Acquisition parameters of mass spectrometry MRM

| Q1 | Q3 | RT | Compound | DP | CE | CXP |
|---|---|---|---|---|---|---|
| 315.8 | 298.1 | 0.968 | Glucose_1 | 92 | 8 | 10 |
| 315.8 | 262.3 | 0.968 | Glucose_2 | 92 | 13 | 9 |
| 316.2 | 298.1 | 0.966 | Fructose_1 | 97 | 9 | 10 |
| 316.2 | 279.8 | 0.966 | Fructose_2 | 108 | 12 | 9 |
| 316.1 | 298.1 | 0.959 | Galactose_1 | 60 | 8 | 10 |
| 316.1 | 208.1 | 0.959 | Galactose_2 | 60 | 13 | 7 |
| 316.3 | 298.2 | 0.945 | Mannose_1 | 96 | 8 | 10 |
| 316.3 | 208.2 | 0.945 | Mannose_1 | 96 | 15 | 7 |
| 224 | 151.9 | 1.21 | Lactate_1 | −138 | −20 | −8 |
| 224 | 137.1 | 1.21 | Lactate_2 | −135 | −25 | −4 |

### 5. Data Analysis

The software MultiQuant is used for integration, and standard curve is used for content calculation.

### 5.1 Standard Curve

### 5.1.1 Preparation of Standard Solution

(1) 20 mg of standard substance is weighed accurately and dissolved in 1 mL of 50% acetonitrile solution;
(2) the standard and the prepared 3-NPH (200 mM) and EDC (120 mM, containing 6% pyridine) solution (2:1:1, v/v/v) are vortexed for 1 min and mixed evenly (total volume of 1 mL);
(3) reaction at 40° C. for 1 h, followed by shaking once every 5 min;
(4) the single standard is derivatized firstly, and the mixed standard is prepared with 50% acetonitrile after the derivatization is completed;
(5) 50% acetonitrile (diluted) is used to prepare a standard series solution with suitable concentration. Standard solutions are prepared on the spot.

### 5.1.2 Plotting of Standard Curve

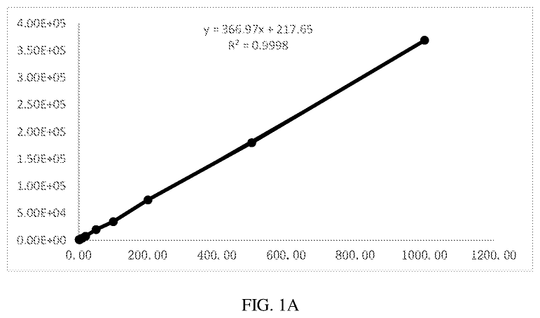

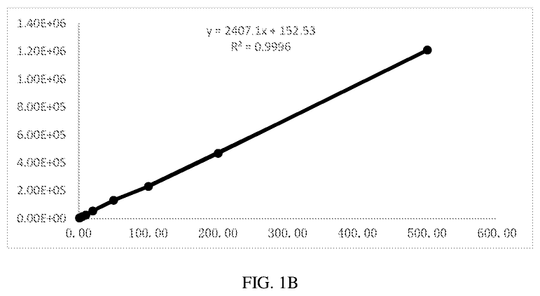

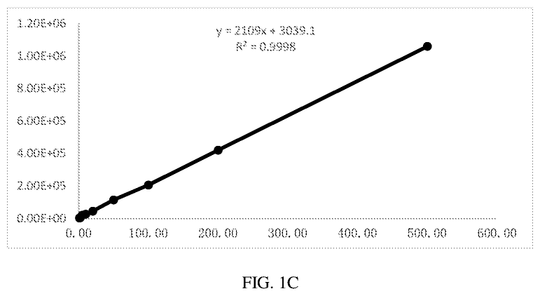

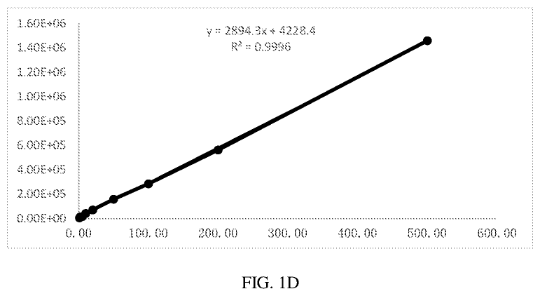

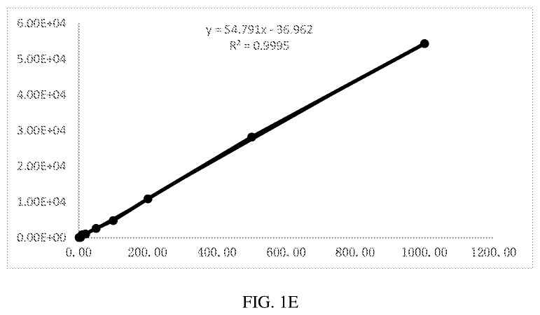

Linear regression is conducted with the concentration of short-chain fatty acids as the abscissa and the peak area of short-chain fatty acids as the ordinate, and R² is greater than 0.99. As can be seen from the details as shown in FIG. 1A to FIG. 1E, the standard curve has a good linear relationship and may be used to calculate the concentration of five substances (glucose, fructose, galactose, mannose and lactate) in the sample.

### 6. Statistical Analysis

Continuous variables are expressed by mean±standard deviation (SD), and independent sample t test or one-way analysis of variance (ANOVA) and false discovery rate (FDR) are used for comparison and correction. The classified data are evaluated by ×2 test. Pearson correlation analysis is used to analyze the correlation between blood sugar level and monosaccharide and lactate levels in tear fluid. The receiver operator characteristic (ROC) curves are analyzed to evaluate the sensitivity and specificity of monosaccharide and lactate in predicting diabetic retinopathy and proliferative diabetic retinopathy. All statistical analyses are conducted using commercially available software (IBM SPSS Statistics 22; SPSS Inc., Chicago, IL), and p<0.05 is considered a statistically significant difference.

### 7. Results

(1) Contents of five metabolites (glucose, fructose, galactose, mannose and lactate) in non-diabetic control group, non-proliferative diabetic retinopathy group and proliferative diabetic retinopathy group.

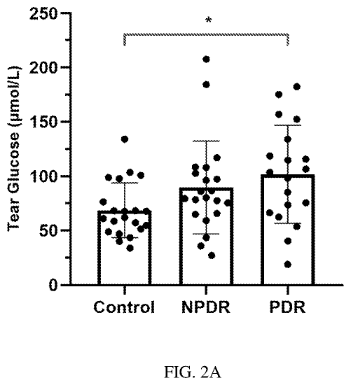

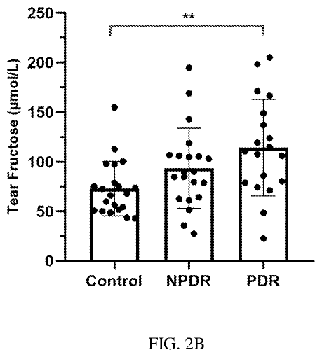

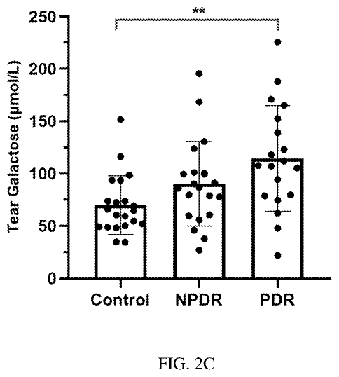

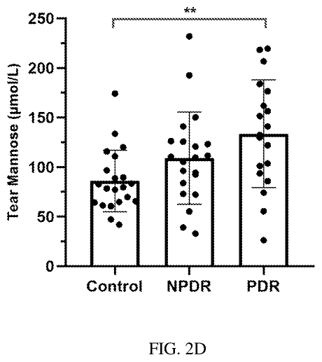

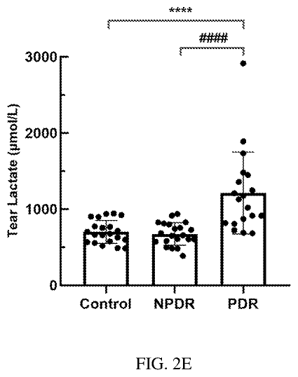

As shown in FIG. 2A-FIG. 2E, the concentrations of five metabolites in tear fluid gradually increases with the progress of the disease, and the increase of lactate content in proliferative diabetic retinopathy is the most statistically significant.
(2) It is found among the diabetic retinopathy patients that the blood glucose level is linearly correlated with the glucose, fructose, galactose and mannose levels in tear fluid.

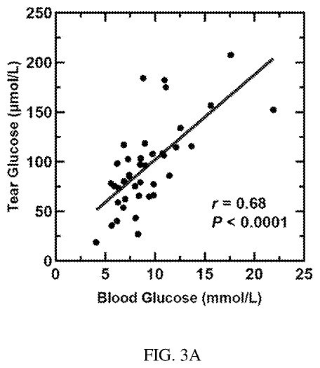

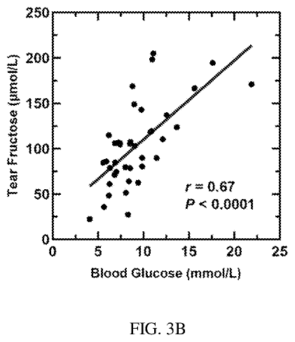

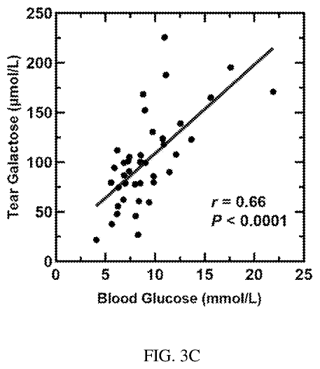

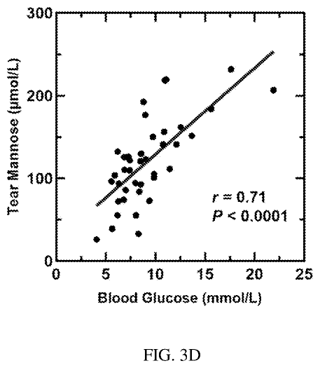

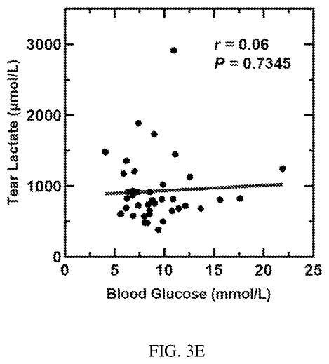

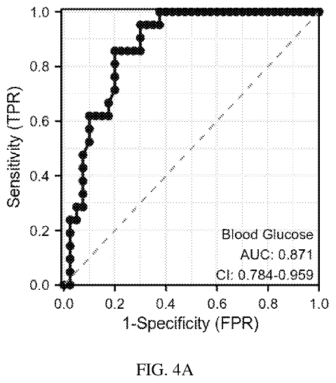

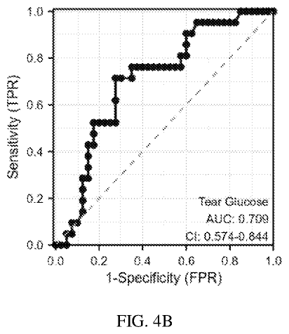

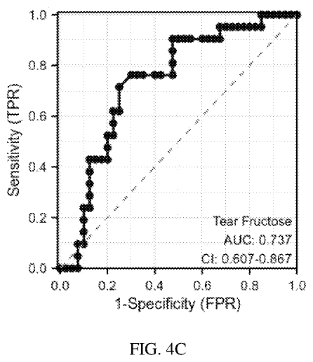

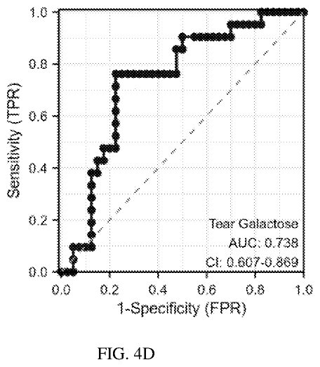

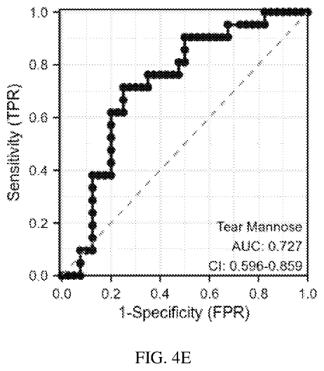

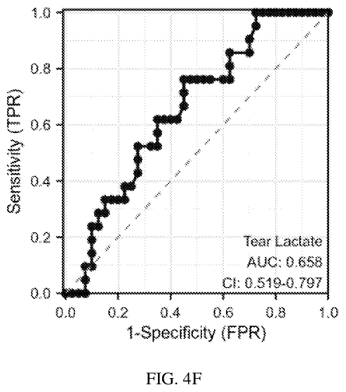

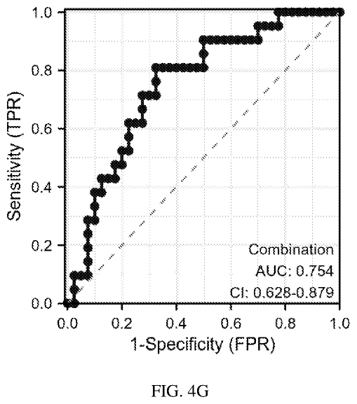

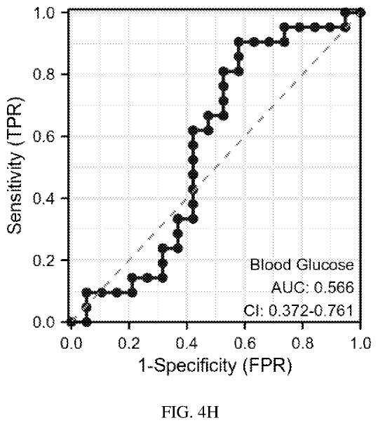

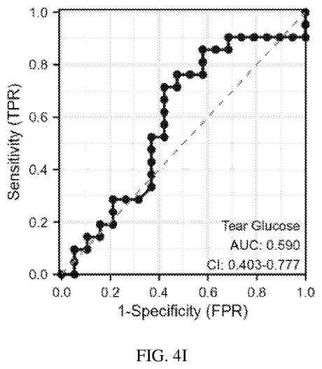

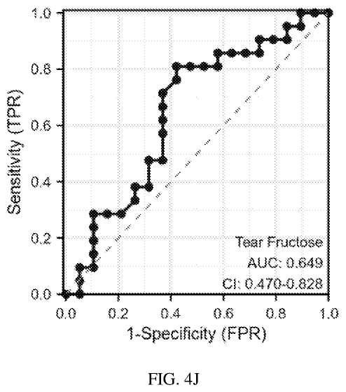

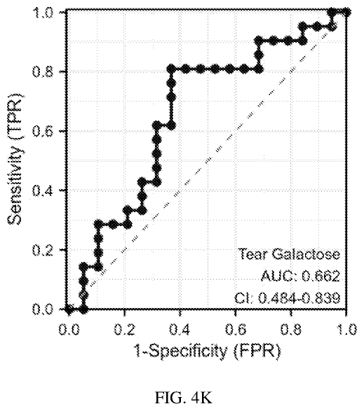

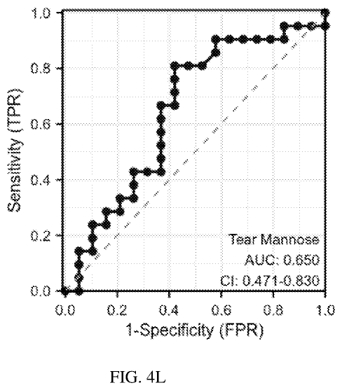

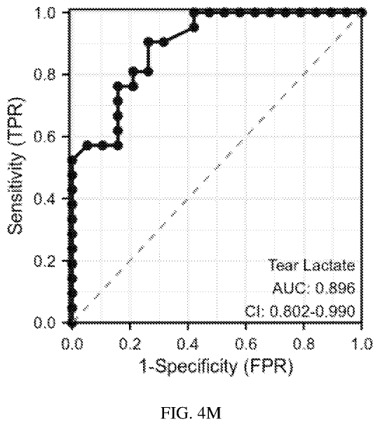

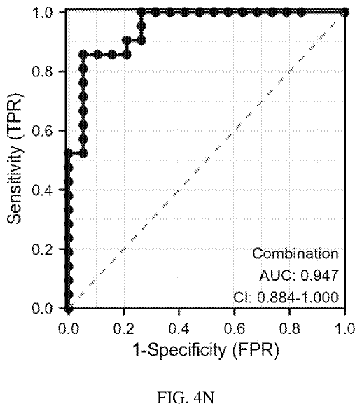

As shown in FIG. 3A-FIG. 3E, the blood glucose level is highly linearly correlated with the mannose level in tear fluid, moderately correlated with the glucose, fructose and galactose levels in tear fluid, and basically has no correlation with the lactate level. It is suggested that lactate is an independent risk factor, independent of the regulation of blood sugar level.
(3) The prediction results of diabetic retinopathy and proliferative diabetic retinopathy are illustrated in FIG. 4A to FIG. 4N, with the ROC curve results showing that:
\textcircled{1} the area under the curve (AUC) for blood glucose for discriminating diabetic retinopathy from non-diabetic subjects is 0.871, and the AUCs for glucose, fructose, galactose, mannose, and lactate in tear fluid are 0.709, 0.737, 0.738, 0.727, and 0.658, respectively; and for the combination of the five components of the tear fluid, the AUC is 0.754.
\textcircled{2}) The AUC of blood glucose for discriminating PDR and NPDR is 0.566, and the AUCs of glucose, fructose, galactose, mannose, and lactate in tears are 0.590, 0.649, 0.662, 0.650, and 0.896, respectively; and the AUC for the combination of the five components of the tear fluid is 0.947.

The above results suggest that the tear lactate may be a potential new biomarker to assess proliferative diabetic retinopathy; tear glucose, fructose, galactose, mannose, and lactate may be a potential new combination of biomarkers to assess proliferative diabetic retinopathy and predict diabetic retinopathy progression.

The above-mentioned embodiments only describe the preferred mode of the present disclosure, and do not limit the scope of the present disclosure. Under the premise of not departing from the design spirit of the disclosure, various modifications and improvements made by ordinary technicians in the field to the technical scheme of the present disclosure shall fall within the protection scope determined by the claims of the present disclosure.

1. Reagents or kits for the early diagnosis and prediction of diabetic retinopathy progress, wherein the reagents or the kits are prepared using glycometabolomics-related biomarkers, and wherein:
glycometabolomics-related biomarkers include one of a lactate, and a combination of the lactate with at least one monosaccharide,
the diabetic retinopathy comprises proliferative diabetic retinopathy and non-proliferative diabetic retinopathy,
the reagents or kits are reagents or kits for detecting the glycometabolomics-related biomarkers in tear fluids and
the glycometabolomics-related biomarkers in the tear fluids are detected based on Ultra-performance liquid chromatography-tandem mass spectrometry (UPLC-MS/MS).

2. (canceled)
